(19) United States
(12) Patent Application Publication
(10) Pub. No.: US 2006/0210520 A1
(43) Pub. Date: Sep. 21, 2006

(54) Macadamia lipid based surfactants and derivatives and process for preparing same

(75) Inventors: Samad A. Syed, Paramus, NJ (US); Ismail I. Walele, Saddle Brook, NJ (US)

Correspondence Address: WEINGRAM & ASSOCIATES P.C., P.O. BOX 927, MAYWOOD, NJ, 07607, US

(73) Assignee: Finetex, Inc.

(21) Appl. No.: 11/081,788

(22) Filed: Mar. 16, 2005

(51) Int. Cl. A61K 8/44

(52) U.S. Cl. 424/70.21; 554/52

(57) ABSTRACT

Novel macadamia-lipid based esters, derivatives, surfactants and emollients are disclosed, as well as their use in the production of surface active derivatives or non-surface active esters and ester emollients. The esters, surfactants and fatty derivatives are useful as emollients, dispersants, emulsifiers, and conditioners for hair care and skin care products. For example, derivatives such as amides of Macadamia fatty triglycerides (“MFTG”)/Monoethanolamine; Acyl Amidopropyl Dimethyl Amines of MFTG as cationic surfactants; Betaines based on MFTG; Esters of MFTG based Fatty Acids and Isostearyl alcohol or PPG-3 Myristyl Ether or Ethoxylated C12-C15 Alcohol or Ethoxylated cetearyl alcohol; Quaternaries based on MFTG and Amino-propyl Dimethylamine; Sulfosuccinates based on MFTG-MEA Amides; and sulfosuccinates based on MFTG-Isopropanolamines have been prepared. Derivatives may also include those involving other reactive groups known to those skilled in the art.

### BACKGROUND OF THE INVENTION

1. Field of the Invention

The present invention relates to novel Macadamia-lipid based surfactants and derivatives, and emollients, their process of manufacture, their use in the production of surface active derivatives, non-surface active esters, and ester emollients, and skin and hair care preparations containing the surfactants, derivatives and emollients. The surfactants and fatty ester derivatives are useful as emollients, dispersants, emulsifiers and conditioners for hair care and skin care products.

2. Description of the Related Art

Surfactants and derivatives are known for a variety of different applications for cosmetic, pharmaceutical, and medicinal purposes.

Numerous references describe the production and use of surface active derivatives or non-surface active esters, and ester emollients. For example, it is known to use macadamia oil in cosmetic preparations to obtain the benefits of mildness. Surfactants and fatty derivatives useful as emollients, dispersants, emulsifiers and conditioners for hair and skin care products are commonly produced from a wide variety of fatty acids, fatty alcohols and amines, polyamines, dialkyl propylamines, alkanolamines, etc. However, none of these references teach or suggest the specific novel macadamia-based surfactants made from the fatty oil triglyceride obtained from macadamia nuts, or MFTG, of this invention or the use of MFTG to produce surface active derivatives or non-surface active esters, and ester emollients for cosmetics and personal care products.

More specifically, macadamia-based compositions are disclosed in U.S. Pat. No. 5,653,966 to Bertoli et al. discloses a lipid composition for cosmetic products comprising, in part, macadamia oil. The preparation of the lipid composition includes refining the oils.

U.S. Pat. No. 6,464,991 to Walele et al. discloses oat-lipid based derivatives, surfactants and emollients and their use in the production of surface active derivatives, or non-surface active esters, and ester emollients.

U.S. Pat. No. 6,599,936 to Bajor et al. discloses cosmetic skin care methods and compositions containing methyl branched esters. The cosmetic compositions provide control of sebum secretion from sebocytes which cause oily skin, and improved skin feel and skin anti-aging benefits. Example 8 identifies macadamia oil as one ingredient in a cosmetic water-in-oil emulsion.

However, among the foregoing patents, none disclose or suggest the specific novel macadamia lipid based surfactants and derivatives of the invention, a process for preparing same, or the use of such derivatives and surfactants having mildness and emolliency for cosmetics and personal care products.

### OBJECTS AND SUMMMARY OF THE INVENTION

It is an object of the invention to produce novel Macadamia-based surfactants and derivatives that are made from a naturally renewable source.

It is another object of the present invention to provide surfactants and derivatives that are made from Macadamia fatty triglycerides.

It is another object of the invention to provide novel Macadamia-based surfactants and derivatives for use in hair care, skin care and other personal care products.

It is yet another object of the invention to provide novel Macadamia-based surfactants and derivatives which have a usefulness similar to conventional amides and betaines.

A further object of the invention is to provide novel Macadamia-based surfactants and derivatives for use in skin care products where the skin feel is even better than conventional derivatves.

It is a further object of the invention to provide Macadamia-based surfactants and derivatives having mildness and emolliency for use in skin care and hair care products. A still further object of the invention is to provide novel Macadamia-based surfactants and derivatives having a fatty acid composition profile close to that of the principal constituents of the human skin surface lipids that play a key role in maintaining the normal barrier function of healthy skin.

Yet another object of the invention is to provide novel Macadamia-based surfactants and derivatives having very low to negligible toxicological effects.

Another object of the invention is to provide formulations containing novel Macadamia-based surfactants and derivatives that are very mild and have a very low toxicological profile.

Another object of the invention is to provide low-irritation Macadamia-based surfactants and derivatives for skin care and hair care products.

Another object of the invention is to provide a method of producing mild, emollient, hair care or skin care products using Macadamia-based surfactants and derivatives.

These and other objects are accomplished by providing Macadamia-based surfactants and derivatives that are improved as compared to commercially available surfactants and derivatives, as they are made from a naturally renewable source, namely, Macadamia oil. The surfactants and derivatives are mild; skin and hair care formulations containing the surfactants and derivatives of the invention are mild and emollient, have very low toxicological profiles, and are not irritating to the skin.

### DETAILED DESCRIPTION OF THE INVENTION

The major fatty raw materials used in the process of the invention are fatty triglycerides derived from sources that are naturally renewable, such as coconut oil, soybean oil, sunflower oil, corn oil, and canola oil, castor oil, etc. A preferred source is Macadamia oil extracted from Macadamia nuts.

The surfactants, derivatives, and emollients of the invention are made from fatty oil triglycerides obtained from the extraction of the kernel of Macadamia Nuts.

Macadamia fatty triglycerides (hereinafter referred to as “MFTG”) are essentially comprised of saturated and unsaturated fatty acids. MFTG comprises approximately 80% monounsaturated acids in its triglycerides, of which 21% is Palmitoleic Acid, a naturally occurring skin lipid that is similar to the sebum of human skin. No other known plant oil has a similar composition. The high level of Palmitoleic acid in MFTG enhances its affinity with natural skin lipids and is the key ingredient in anti-aging cream designed to help maturing skin retain its softness, resilience, and youthful appearance. Due to its exceptional composition, it has better moisturizing properties and is very effective in hydrating the lower (dermal) layers of the skin. This type of moisturization will also respond to skin conditions such as eczema, psoriasis and itching of dry skin caused by repeated washing.

The composition of Macadamia fatty acid triglycerides is as follows:

| | |
|---|---|
| Palmitic acid | 7.0%-9.5% |
| Palmitoleic acid | 15%-22% |
| Stearic acid | 2.0%-5.5% |
| Oleic acid | 55%-67% |
| Linoleic acid | 1.0%-4.0% |

Surfactants and fatty derivatives useful as emollients, emulsifiers and conditioners for hair and skin care products are commonly produced from a wide variety of fatty acids, fatty alcohols and amines, polyamines, dialkylpolyamines, alkanolamines, etc. Unless otherwise specified herein, the term “derivatives” is a broad term meant to include surfactants, nonsurface active esters, and ester emollients.

The following list of reactants and their respective derivatives such as esters, amides, betaines, quaternaries, etc, is exemplary of the type of reactants and derivatives on which the emollient surfactants and derivatives may be based, and as such, is not to be considered limiting.

Group A (Reactants): Fatty acids or methyl esters of fatty acids or fatty oils sourced from naturally renewable fatty triglycerides of Macadamia nuts.

Group B (Reactants) may be selected from the group consisting of: Alkanolamines such as Monoethanolamine and Diethanolamine; Dialkylaminopropylamines such as Dimethyl Aminopropylamine; Polyamines such as Diethylene triamine, Triethylene tetraamine; primary and secondary amines; and fatty alcohols (linear and/or branched) (C8-C22 alcohols) (ethoxylated or propoxylated).

The preferred reactants used in preparing the surfactants and derivatives of the invention are selected from the group consisting of:
A. MFTG and Monoethanolamine, which are reacted to form a product which is Macadamia amide-MEA.
B. MFTG and Diethanolamine.
C. MFTG and Monoisopropanolamine.
D. MFTG and Diisopropanolamine.
E. MFTG and Dimethylaminopropylamine, which are reacted to form a product which is Macadamiaamidopropyldimethylamine.
F. Reaction product of E. (i.e., Macadamiaamidopropyldimethylamine) and Sodium Monochloroacetate, which are reacted to form a product which is Macadamiaamidopropylbetaine.
G. Reaction product of E. (i.e., Macadamiaamidopropyldimethylamine) and Diethylsulfate which are reacted to form a product which is Macadamiaamidopropyldimethyl ethyl Ethosulfate.
H. Reaction product of Macadamia amide MEA-Maleate (obtained from Macadamia amide-MEA and maleic anhydride) and sodium sulfite, which are reacted to form a product which is Disodium Macademia-amide MEA-Sulfosuccinate.
I. MFTG Methylated (i.e., MFTG and Methanol giving methylmacadamiate) and Isostearyl alcohol which are reacted to form a product which is Isostearyl Macadamiate.
J. MFTG Methylated and PPG-3 Myristyl Ether which are reacted to form a product which is PPG-3 Myristyl Macadamiate.
K. MFTG Methylated and Ethoxylated C12-C15 Alcohol which are reacted to form a product which is C12-15 Pareth-7 Macadamiate.
L. MFTG Methylated and Ethoxylated Cetearyl alcohol which are reacted to form a product which is Ceteareth-4 Macadamiate.

MFTG is used in either exact stoichiometric proportions or in some excess over Reactant B.

In specific embodiment, and by way of illustration, this invention contemplates the production of the following macadamia lipid-based esters, surfactants and derivatives:
1. Macadamia-amide MEA is the reaction product of MFTG and Monoethanolamine.

It has the following structure: 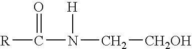
Where R=Macadamia fatty acids group
2. Macadamia amide-MEA and sodium methyl cocoyl taurate is a blend or mixture of Macadamia-amide MEA and sodium methyl cocoyl taurate: 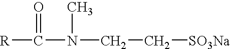
   Where R=macadamia fatty acids group.
3. Macadamiaamideopropyldimethylamine is the reaction product of MFTG and Dimethylaminopropylamine. It has the following structure:

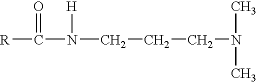

Where R=macadamia fatty acids group
4. Isostearyl Macadamiate is the reaction product of MFTG Methylated and Isostearyl alcohol. It has the following structure: 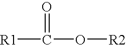
   Where R1−Macadamia fatty acids group
   R2=Isostearyl Alcohol group
5. PPG-3 Myristyl Macadamiate is the reaction product of MFTG Methylated and PPG-3 Myristyl Ether. It has the following structure: 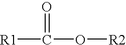
   Where R1=Macadamia fatty acids group
   R2=PPG-3 Myristyl Ether group
6. C12-15 Pareth-7 Macadamiate is the reaction product of MFTG Methylated and C12-15 Pareth-7 (e.g., Neodol 25.7). It has the following structure: 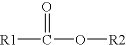
   Where R1=Macadamia fatty acids group
   R2=C12-15 Pareth-7 alcohol group
7. Ceteareth-4 Macadamiate is the reaction product of MFTG Methylated and Ceteareth-4 alcohol. It has the following structure: 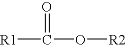
   Where R1=Macadamia fatty acids group
   R2=Cetereath-4 Alcohol group
8. Macadamia amidopropylbetaine is a betaine of Macademiaamidopropyldimethylamine having the following structure: 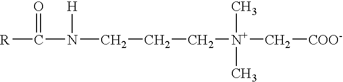
   Where R=Macadamia fatty acids group
9. Disodium Macadamia-MEA-sulfosuccinate is the reaction product of Macadamia amide MEA-Maleate and sodium sulfite. It has the following structure: 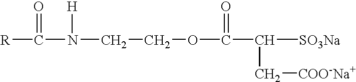
   Where R=Macadamia fatty acids group
10. Macadamia amido propyl dimethyl ethyl ethosulfate is a reaction product of Macadamia amido propyl dimethyl amine and diethylsulfate. It has the following structure: 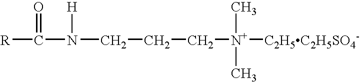
   Where R=Macadamia fatty acids group

The above derivatives have shown usefulness similar to conventional amides and betaines with the additional benefits of being mild to the skin. Additionally, skin feel is even better than when using conventional derivatives.

Some of these surfactants and derivatives are used in skin care and hair care formulations and dermatological preparations. Personal care formulations prepared according to the invention, which contain the surfactants and derivatives added thereto, can be formed, without limitation, into applications such as solutions, emulsions, gels, solids, emulsions, aerosols, powders, creams, granules, or tablets.

Mildness and emolliency of these derivatives is of importance to the improved skin and hair care products including the surfactants and derivatives of the invention. A major aspect of derivatives of this invention is their very low to negligible toxicological effects indicating their mildness to skin care products.

Thus, the advantages of the derivatives of the invention include:
Made from naturally renewable source
Mildness of the derivatives
Mildness of the formulations containing the derivatives
Very low toxicological profile
Low irritation

Thus, the novel macadamia oil-based surfactants and derivatives of this invention have unique properties in that they are mild to the skin and have low to negligible toxicological effects. These properties make these derivatives useful as a vehicle or carrier, dispersants, emulsifiers, emollient, solubilizer and conditioners for skin care and hair care formulations such as hair creams, hand cleaners, bath oils, suntan oils, anti-perspirants, perfumes, colognes, cold creams, electric pre-shaves, eye and throat oils, finger nail polish, topical pharmaceutical ointments, lipsticks, stick rouge, skin lotions and creams, skin moisturizers, cleansing creams, and after-bath splash and lotions, as well as other formulations. The foregoing list is only exemplary of the type of compositions in which the macadamia-based surfactants and derivatives of this invention may be used, and, as such, is not to be considered limiting.

The amount of such macadamia oil-based surfactants and derivatives to be used in such compositions is dependent on the type of hair care and skin care compositions, the desired dosage or amount of active ingredient to be delivered, the type and quantity of other ingredients, such as cosmetic ingredients used, the amount and type of functional additives that are utilized, the user's skin and hair type, and the severity and extent of the skin or hair condition, and other parameters that will be apparent to those skilled in the art. Generally, compositions containing the macadamia-based surfactants and derivatives of the invention are topically applied in effective amounts to the affected areas of the skin or to hair. Typically, the amount of surfactants and derivatives used ranges from about 1.0% to about 30.0%, by weight, of the skin care compositions. For example, a facial cream may only have about 5.0%, while a massage oil may have up to about 20% by weight. Still higher amounts may be used in, for example, bath oils, e.g. 50%.

Further, the macadamia-based surfactants and derivatives of this invention possess other unusual physiochemical properties, which can make them suitable for use as emollient carriers in cosmetic formulations, and for use as solvents and emollient carriers in general cleaning compositions, such as in hand, face, and body creams and lotions. Thus, the macadamia-based surfactants and derivatives described herein may serve not only as emollients and carriers, but may also exhibit one or more other functions.

The surfactants and derivatives of the invention have properties such as, being less greasy, less oily, low toxicity, ease of emulsification, acid and alkaline stability, the ability to form gels with suspending agents, water solubility/dispersibility, and the ability to act as solvents for many common skin and hair care ingredients.

The following are non-limiting examples of processes for preparing the surfactant and derivative compositions of the invention (Examples 1 to 15), analytical results for several of said compositions (Examples 16 to 18), as well as uses of the compositions in specific cosmetic or personal care product formulations (Examples 19 to 38). In the Examples, as well as throughout this application, the chemical and scientific symbols have their customary meanings and all percents are weight percents unless otherwise specified.

Example Nos. 1 through 15 identify surfactants and derivatives produced by the process of the invention. For ease of identification, each ester is identified by both an Example Number and a Reference No., where applicable. This identification system is used in the subsequent Tables.

Although the Examples use only selected compounds and formulations, it should be understood that the examples are illustrative and not limited. Thus, any of the aforementioned Reactants A and B may be substituted according to the teachings of this invention in the following Examples.

### EXAMPLE # 1

### Ref. No. 137-21

### Preparation of Monoethanolamide of Macadamia Fatty Triglyceride

(Macadamia Amide—MEA)

182.38 gms. of Macadamia fatty triglycerides (MFTG), also called Macadamia fatty oil (available under the trade name of Oil of Macadamia from Macadamia Oil of Australia, N. South Wales, Australia) were charged to a one liter round bottom 4-neck flask with distillation assembly. To this was added 38.10 gms. of Monoethanolamine (MEA) and 4.52 gms. Sodium Methylate 25% w/w in methanol. Reaction mass was heated under nitrogen to between 105° C. and 120° C. and was kept over a period of 4 hours until the alkalinity was about 20 mg KOH/g. This was a light yellow material that solidified upon cooling. The total mass was collected 225 gms. The distillate of free methanol was approximately 1.25 gms.

### EXAMPLE #2

### Ref. No. 137-30

### Preparation of Fluid Delivery System for Macadamia-Amide-MEA of Example #1

(Macadamia Amide MEA and Sodium Methyl Cocoyl Taurate)

The solid amide of Example #1 (Macadamia-Amide-MEA) is blended with an aqueous solution of Sodium Methyl Cocoyl Taurate resulting in a fluid, flowable dispersion paste. Thus, 8 gms. of Macadamia-Amide-MEA of example #1 was mixed at 70° C. with a mixture of 70 gms. of Sodium Methyl Cocoyl Taurate (Tauranol WS conc. from Finetex, Inc. of Elmwood Park, N.J.) and 22 gms. of water. A smooth off-white paste with very good flow was obtained upon cooling with mixing.

### EXAMPLE #3

### Ref. No. 137-23

### Preparation of Amido Amine of MFTG and Dimethyl Amino Propyl Amine

(Macadamia-amidopropyl Dimethylamine)

A reaction flask of 500 ml. capacity, with distillation assembly and mixer, was set up under nitrogen. To this was added 213 gms. of Macadamia fatty triglyceride (MFTG) also called Macadamia fatty oil available under the trade name of Oil of Macadamia from Macadamia Oil of Australia, N South Wales, Australia. Heated to 70° C. under nitrogen. To this was added 81 gms. of Dimethylaminopropylamine (DMAPA) and 6.0 gms. Sodium Methylate 25% in methanol. Temperature was brought to 120° C.-125° C. over 45 minutes. Heat input was adjusted to reach 150° C. over the next 30-45 minutes. Reaction was continued at 145° C.-150° C. for 4 hrs with use of vacuum. Reduced pressure ranging from 5 to 22 mm Hg. was used progressively to remove the free DMAPA. The yield upon cooling was 270 gms. The distillate collected was 30 gms. which was almost pure DMAPA. The final product had an alkalinity of 170 mg KOH/g.

### EXAMPLE # 4

### Ref. No. 137-24

### Preparation of Betaine Derivative from Intermediate of Example #3 (i.e., Macadamia-amidopropyl Dimethylamine)

(Macadamia-amidopropylbetaine)

To a 500 ml. reaction flask was added 189.93 gms. water and 22.14 gms. Sodium Monochloroacetate (SMCA). All SMCA was dissolved at 25° C. To this was added 37.29 gms. of 1,3 Butylene glycol. A clear liquid was formed. The premelted 47.64 gms. of the product of Example #3 were added to the mixture at 70° C.; the addition was done in three equal portions. These additions were done at 85° C.-86° C. and over a period of approximately one hour. The reaction mass was then held at 85° C. for further two hrs. The alkalinity was found to be 4.95 mg KOH/g and acid value was almost none. A clear colorless liquid with 4.0% sodium chloride and 53.0% of water was obtained. The net yield was 300 gms. as a Betaine.

### EXAMPLE # 5

### Ref. No. 137-26

### Preparation of Macadamia Amidopropyl Dimethyl Ethyl Ethosulfate

In a four neck 500 ml. flask equipped with stirrer, condenser, nitrogen inlet and thermometer was added, under nitrogen, 105 gms. of Macadamia amidopropyl dimethyl amine (product of Example #3) and 120 gms. of water and 31.12 gms. butylene glycol. This was mixed and the mixture was brought to 60° C. To this mixture at 60° C. was added 43.80 gms. of Diethyl sulfate. Reaction mixture was kept at between 90° C.-105° C. Any exotherm was controlled by manipulating the heating mantle to maintain temperature below 100° C. Reaction continued for about 3 hrs, when alkalinity was 3.5 mgs KOH/gm. The clear colorless liquid so obtained was completely water soluble and foamed heavily upon dilution with water. The anhydrous yield was 280 gms.

### EXAMPLE # 6

### Ref. No. 137-28

### Preparation of Disodium Macadamia-Amide-MEA Maleate Intermediate

To a 4 neck round bottom flask of 500 ml. capacity equipped with stirrer, condenser, and nitrogen inlet was added 63.18 gms. of Macadamia amide MEA (i.e. Product of Example #1). Heated mildly to 70° C. Added 18.27 gms. of Maleic anhydride. Reaction exothermed to 80° C. Mixture was held at 85° C. for one hour, acidity was found to be 122 mgm. KOH/gm. This was discharged as a clear colorless liquid and the net yield weighed at 81.45 gms.

### EXAMPLE # 7

### Ref. No. 137-28 B

### Preparation of Disodium Macadamia-Amide-MEA-Sulfosuccinate

A 500 ml. round bottom flask equipped with condenser, Nitrogen inlet and stirrer was charged with 195.09 gms. of water and 23.49 gms. of sodium sulfite. The sulfite crystals were completely dissolved. To this was added 81.45 gms. of Macadamia Amide-MEA-Maleate intermediate (product of Example #6). The reaction mixture was held at 85° C. with the controlled addition of the Macadamia amide-MEA-Maleate intermediate. The mixture was gelled initially which fluidized as the reaction continued with the conversion. The yield was 300 gms. net in the flask with pH of 6.5 and showed 35% solids. It was a thick dispersion. A further dilution with additional 40 gms. of water was done. A fluid light yellow dispersion was obtained. This product gave a very good rich foam when dissolved in water and further diluted for washing hands. The skin feel was also very smooth and pleasant without defatting effect.

### EXAMPLE # 8

### Ref. No. 137-25

### Preparation of Methyl Ester of Macadamia Fatty Triglycerides (MFTG)

880.7 gms. of Macadamia fatty triglycerides (MFTG) also called Macadamia oil was charged to a 2 liter round bottom 4-neck flask with distillation assembly under nitrogen. Heated to 82° C. To this was added 109.3 gms. of methanol and 2.0 gms. of Potassium Hydroxide flakes. Reaction mass then mixed at 82° C. for 30 minutes. Allowed to stand for 4 hours and the bottom glycerine layer was removed. The top layer, upon further standing, was a clear liquid methyl ester of Macadamia fatty oil. The residue from the methyl ester layer was approximately 280 gms. The yield as methyl ester was 780 gms. of the top clear liquid. This was further subjected to distillation for removal of methanol, which collection was approximately 40 gms. The finished filtered yield of the methyl ester was 740 gms.

### EXAMPLE # 9

### Ref. No. 137-34

### Isostearyl Ester of MFTG Fatty Acids

(Isostearyl Macadamiate)

112.80 gms. of Methyl ester of example # 8 was charged to 500 ml. reaction flask with distillation assembly. To this was added, under nitrogen, 127.20 gms. of Isostearyl alcohol and 5.0 gms. of Sodium Methylate 25% in methanol. The mass was reacted at 150° C. for approximately 2 hours and any free methanol or methanol of rection was removed by distillation at reduced pressure of 10 Hg. The methanol distillate collected was approximately 5.0 gms. with some leftover condensate in the condenser. The mass was further refurbished with 3.0 gms. of Sodium Methylate and further reacted at 160° C., with vacuum of 15 mm Hg. The ester was then collected which showed solidification characteristics. The 240 gms. of Isostearyl ester was then washed with 50 gms. of water containing 5.0 gms. Sodium chloride and 1.0 gm. of concentrated Hydrochloric acid adjusted to pH 6. Separation occurred at 75° C. Allowed to stand. Separated bottom layer weighing 60 gms. A second wash was given with 50 gms. water and 5.0 gms. Sodium chloride. Bottom aqueous layer of 60 gms. was drained and the top layer of the ester collected was 225 gms. This wet ester was then dried in the reaction flask at 125° C. at reduced pressure of 25 Hg. The ester was then collected and filtered using diatomaceous earth. A liquid of light yellow color was obtained. The net filtered yield was 225 gms.

### EXAMPLE # 10

### Ref. No. 137-36

### PPG-3 Myristyl Ether Ester of MFTG Fatty Acids

(PPG-3 Myristyl Ether Macadamiate)

123 gms. of Methyl ester of example #8 was charged to a 500 ml. reaction flask with distillation assembly. To this was added, under nitrogen, 177 gms. of PPG-3 Myristyl Ether and 5.0 gms. of Sodium Methylate 25% in methanol. The mass was reacted at 150° C. for approximately 2 hours and any free methanol or methanol of reaction was removed by distillation at reduced pressure of 10 Hg. The methanol distillate collected was approximately 5.0 gms. with some leftover condensate in the condenser. The mass was further refurbished with 3.0 gms. of Sodium Methylate and further reacted at 160° C. with vacuum of 15 mm Hg. The ester was then collected which showed solidification characteristics. The 285 gms. of PPG-3 Myristyl Ether ester was then washed with 50 gms. of water containing 5.0 gms. sodium chloride and 1.0 gms. of concentrated Hydrochloric acid adjusted to pH 6. Separation occurred at 75° C. Allowed to stand. Separated bottom layer weighing 60 gms. A second wash was given with 50 gms. water and 5.0 gms. Sodium chloride. Bottom aqueous layer of 60 gms. was drained and the top layer of the ester collected was 225 gms. This wet ester was then dried in the reaction flask at 125° C. at reduced pressure of 25 Hg. The ester was then collected and filtered using diatomaceous earth. A liquid of light yellow color was obtained. The net filtered yield was 225 gms.

### EXAMPLE # 11

### Ref. No. 137-76

### C12-C15 Ethoxylated Ester of MFTG Fatty Acids

(C12-15 Pareth-7 Macadamiate)

112.80 gms. of Methyl ester of example # 8 was charged to 500 ml. reaction flask with distillation assembly. To this was added under nitrogen 141.20 gins. of C12-15 Pareth-7 and 5.0 gms. of Sodium Methylate 25% in methanol. The mass was reacted at 150° C. for approximately 2 hours and any free methanol or methanol of reaction was removed by distillation at reduced pressure of 10 Hg. The methanol distillate collected was approximately 4.0 gms. with some leftover condensate in the condenser. The mass was further refurbished with 3.0 gms. of Sodium Methylate and further reacted at 160° C. with vacuum of 15 Hg. The ester was then collected which showed solidification characteristics. The 250 gms. of crude ester was then washed with 50 gms. of water containing 5.0 gms. sodium chloride and 1.0 gms. of concentrated Hydrochloric acid adjusted to pH 6. Separation occurred at 75° C. Allowed to stand. Separated bottom layer weighing 60 gms. A second wash was given with 50 gms. water and 5.0 gms. Sodium chloride. Bottom aqueous layer of 60 gms. was drained and the top layer of the ester collected was 225 gms. This wet ester was then dried in the reaction flask at 125° C. at reduced pressure of 25 mmHg. The ester was then collected and filtered using diatomaceous earth. A colorless liquid was obtained. The net filtered yield was 220 gms.

### EXAMPLE # 12

### Ref. No. 137-89

### Ethoxylated cetearyl Alcohol Ester of MFTG Fatty Acids

(Ceteareth-4 Macadamiate)

126.39 gms. of methyl ester of example # 8 was charged to 500 ml. reaction flask with distillation assembly. To this was added, under nitrogen, 173.61 gms. of ceteareth—4 and 5.0 gms. of Sodium Methylate 25% in methanol. The mass was reacted at 150° C. for approximately 2 hours and any free methanol or methanol of reaction was removed by distillation at reduced pressure of 10 mmHg. The methanol distillate collected was approximately 7.0 gms. with some leftover condensate in the condenser. The mass was further refurbished with 3.0 gms. of Sodium Methylate and further reacted at 160° C. with vacuum of 15 Hg. The ester was then collected which showed solidification characteristics. The 290 gms. of crude ester was then washed with 50 gms. of water containing 5.0 gms. sodium chloride and 1.0 gm. of concentrated Hydrochloric acid adjusted to pH 6. Separation occurred at 75° C. Allowed to stand. Separated bottom layer weighing 60 gms. A second wash was given with 50 gms. water and 5.0 gms. Sodium chloride. Bottom aqueous layer of 60 gms. was drained and the top layer of the ester collected was 225 gms. This wet ester was then dried in the reaction flask at 125° C. at reduced pressure of 25 mmHg. The ester was then collected and filtered using diatomaceous earth. A liquid of light yellow color was obtained. The net filtered yield was 225 gms.

### EXAMPLE # 13

### Ref. No. 137-38

### Preparation of Monoisopropanol amide of Macadamia Fatty Triglyceride

(Macadamia Amide-MIPA)

234.09 gms. of Macadamia fatty triglycerides (MFTG) also called Macadamia fatty oil (available under the trade name of Oil of Macadamia from Macadamia Oil of Australia, N South Wales Australia) were charged to a one liter round bottom flask with distillation assembly. To this was added 60.09 gms. Monoisopropanol amine (MIPA) and 5.82 gms. sodium Methylate 25% w/w in methanol. Reaction mass was heated under nitrogen to between 105° C. and 120° C. and was kept over a period of 4 hrs until the alkalinity was about 35 mg KOH/g. This was a light yellow material that solidified upon cooling. The total mass was collected 225 gms. The distillate of free methanol was approximately 4.5 gms.

### EXAMPLE # 14

### Ref. No. 137-39A

### Preparation of Disodium Macadamia-Amide-MIPA Maleate

To a 4 neck round bottom flask of 500 ml. capacity equipped with stirrer, condenser, and nitrogen inlet was added 63.18 gms. of Macadamia amide MEA (i.e., product of Example #13). Heated mildly to 70° C. Added 18.27 gms. of Maleic anhydride. Reaction exothermed to 80° C. Mixture was held at 85° C. for one hour. Acidity was found to be 124 mgm. KOH/gm. This was discharged as a light amber liquid and the net yield weighed at 80 gms.

### EXAMPLE # 15

### Ref. No. 137-39 B

### Preparation of Disodium Macadamia-Amide-MIPA-Sulfosuccinate

A 500 ml. round bottom flask equipped with condenser, Nitrogen inlet and stirrer was charged with 195.09 gms. of water and 23.49 gms. of sodium sulfite. The sulfite crystals were completely dissolved. To this was added 81.45 gms. of Macadamia Amide-MIPA-Maleate intermediate (product of Example #14 ). The reaction mixture was held at 85° C. with the controlled addition of the Macadamia amide-MEA-Maleate intermediate. The mixture was gelled initially which fluidized as the reaction continued with the conversion. The yield was 300 gms. net in the flask with pH of 6.5 and showed 35% solids. It was a thick dispersion. A further dilution with additional 40 gms. of water was done. A fluid light yellow dispersion was obtained. This product gave a very good, rich foam when dissolved in water and further diluted for washing hands. The skin feel was also very smooth and pleasant without defatting effects.

The Macadamia-based esters, surfactants and derivatives have the following featured properties:

Ease of emulsification.

Good refractive index.

Emolliency with good after-feel.

Lack of greasiness/pleasant skin feel.

Lack of oiliness while imparting good lubrication.

Low cloud point and pour point.

High spreading coefficient.

Alcohol solubility.

Additive for Antiperspirant formulations.

Low toxicity.

Hydrolytic stability.

Solvent for many skin and hair additives including sunscreens.

The Macadamia based esters, surfactants and derivatives are advantageous in that they are non-oily, tasteless, inert, essentially nontoxic and non-sensitizing, and stable. The Macadamia-based esters, surfactants and derivatives are useful as emollients, solubilizers, moisturizers, plasticizers, sunscreen vehicles/solvents, de-oilers/degreasers, and emulsifiers/co-emulsifiers. The foregoing list is only exemplary of the type of composition in which Macadamia esters may be used and, as such, is not to be considered limiting.

The amount of Ester used in an aqueous surfactant composition depends on the type and quantity of other ingredient used, and the amount and type of functional additives that are utilized. Typically the amount of Macadamia ester used ranges from about 0.5% to about 50% by weight of the aqueous surfactant composition, and preferably, from about 0.5% to about 10% by weight of Macadamia esters are used.

Macadamia-based esters, surfactants and derivatives possess unusual physico-chemical properties. In particular, they possess higher spreading coefficient that can make them beneficial and unique components of sophisticated delivery system such as in hand, face, and body creams and lotions.

Macadamia-based esters, surfactants and derivatives may be used in skin care compositions. The amount used in skin care compositions is dependent on the type of skin care composition, the type and quantity of cosmetic ingredients used and the amount and type of functional additives. Typically, the amount ranges from about 0.5% to about 80% by weight of the skin care composition. For example, a facial cream may only have about 0.5% whereas a massage oil may have up to about 80% by weight. Still higher amounts may be used in, for example, bath oils, e.g., 95%.

Further, the Macadamia-based esters described herein act as solvents and/or vehicles for solid organic, ultraviolet (uv) absorbers. Such esters also function as plasticizers for polymers contained in skin care compositions, and act as auxiliary suspending agents capable of assisting in the suspension of ingredients in skin care compositions and also may function as a dye leveling agents and dye carriers. Thus, the Macadamia based esters, when used in skin care compositions, serves not only as emollients and carriers, but also exhibits one or more other functions.

It is understood that the embodiments described herein are merely exemplary and that a person skilled in the art may make many variations and modifications without departing from the spirit and scope of the invention. All such modifications and variations are intended to be included within the scope of the invention described herein.

### EXAMPLE 16

### Analytical Results

An analysis of the esters was conducted as to appearance, color, surface tension, spreading coefficient, freezing point, percentage of water, refractive index, and specific gravity. The results are presented in Table I below.

TABLE I

| | | PPG-3 | | |
|---|---|---|---|---|
| | | Myristyl | C12-15 | |
| | Isostearyl | Ether | Pareth-7 | Ceteareth-4 |
| Parameters | Macadamiate | Macadamiate | Macadamiate | Macadamiate |
| Form | liquid | liquid | liquid | liquid |
| Freezing Point | −3 | −2 | 10 | 12 |
| (° C.) | | | | |
| Refractive | 1.4700 | 1.4640 | 1.4580 | 1.4540 |
| index (25° C.) | | | | |
| Surface | 31.5 | 31.0 | 32.00 | 32.50 |
| Tension | | | | |
| (25° C.) | | | | |
| Spreading | 32.75 | 34.00 | 31.50 | 32.15 |
| Coeffecient | | | | |
| (° C.) | | | | |
| Viscosity | 40 | 80 | 100 | 120 |
| (CPS) | | | | |
| Color (APHA) | 70 | 50 | 40 | 50 |
| % Water | 0.01 | 0.01 | 0.01 | 0.01 |
| Specific gravity | 1.094 | 1.016 | 1.068 | 1.098 |
| (25° C.) | | | | |
| Interfacial | 7.75 | 7.00 | 8.50 | 8.35 |
| Tension | | | | |
| (25° C.) | | | | |

### EXAMPLE # 17 (Ref No. 137-65)

### Sunscreen Solubilities in Macadamia Based Esters (25° C.)

TABLE II

| | Esters | | | | |
|---|---|---|---|---|---|
| Sunscreen | A | B | C | D | E |
| Benzophenone-3 | 18.36 | 23.01 | 16.22 | 17.4 | 15 |
| Parsol 1789 | 17.89 | 22.23 | 15.55 | 16.2 | 13 |
| Dihydroxyacetone | 2 | <1 | <1 | <1 | 1 |
| Salicylic acid | 2 | 1 | <1 | <1 | 1 |

Where the esters are:
A = Isostearyl Macadamiate
B = PPG-3 Myristyl ether Macadamiate
C = C12-15 Pareth-7 Macadamiate
D = Ceteareth-4 Macadamiate
E = Finsolv TN

The two most commonly used solid organic crystalline sunscreens are Benzophenone-3 (2 hydroxy 4-methoxy benzophenone) and Parsol 1789 (Butyl—methoxy dibenzoyl methane). These two sunscreens are difficult to dissolve and keep in solution for use in sunscreen formulations for optimal SPF (Sun Protection Factor). Higher solvency for a sunscreen active is desirable as it allows higher concentrations of the sunscreen active ingredient in a formulation. This advantageously raises the SPF ratings for the formulations. The liquid organic sunscreens that are commonly used are octylsalicylate (os) and octyl-methoxycinnamate (omc). Macadamia esters exhibit superiority over commonly used and marketed cosmetic emollients/materials.

The high solvency exhibited by the macadamia based esters of the invention for the solid crystalline organic sunscreens is an advantageous effect in formulating sunscreen products for the skincare markets. Thus, besides being cosmetic emollients, these esters are excellent solvents for the above-mentioned organic sunscreens. A further aspect of these esters, beside being solubilizers for sunscreens, is to render anti-washoff effect. This effect is very attractive in formulating long lasting sunscreen products allowing the sunscreen to remain on the skin for a longer period of time.

### EXAMPLE # 18

### Solubility and Compatibility of Macadamia Based Esters

The solubility characteristics of macadamia based esters are tabulated in Table III below. They are soluble in most commonly used solvents, emollients and vehicles employed in cosmetic product formulations.

TABLE III
Solubilities of Esters

| | Macadamiates | | | |
|---|---|---|---|---|
| Solvents & Esters | A | B | C | D |
| Water | −− | −− | −− | −− |
| Ethanol | + | + | + | + |
| Isopropanol | + | + | + | + |
| Mineral Oil | −− | −− | −− | −− |
| Glycerin | −− | −− | −− | −− |
| Propylene Glycol | −− | −− | −− | −− |
| Dimethione Copolyl (Silwet 7604& 7230) | + | + | + | + |
| Dow Corning fluid 344 | + | + | + | + |
| Dow corning fluid 345 | + | + | + | + |
| Isopropyl Myristate | + | + | + | + |
| Isopropyl Palmitate | + | + | + | + |
| Finsolv SLB101 | + | + | + | + |
| Finsolv SLN 201 | + | + | + | + |
| Finsolv PL 62 | + | + | + | + |
| Finsolv PL 355 | + | + | + | + |
| Finsolv BCO 115 | + | + | + | + |
| Finsolv BCR 111 | + | + | + | + |
| Finsolv BOHS 111 | + | + | + | + |
| Finsolv TN | + | + | + | + |
| Finsolv TPP | + | + | + | + |
| Finsolv EMG 20 | + | + | + | + |
| Finsolv BOD | + | + | + | + |
| Finsolv SB | + | + | + | + |
| Finsolv PG22 | + | + | + | + |
| Finester EH 25 | + | + | + | + |
| Finester DOMR | + | + | + | + |

Where the Macadamia based surface active esters are:
A = Isostearyl Macadamiate
B = PPG-3 Myristyl ether Macadamiate
C = C12-15 Pareth-7 ether Macadamiate
D = Ceteareth-4 ether Macadamiate
Key:
−− indicates insoluble
+ indicates soluble

### EXAMPLE # 19

### Ref. No. 137-130

### Soap Bar Evaluation

An evaluation of the use of Macadamia-based esters in bar soaps was conducted as described below. The results show improvements in the properties of bar soaps upon inclusion of the Macadamiate ester. Soap bars containing Syndet base 96-143-1 and combo bar (25% syndet base 96-143-1 and 75% Bradford soap base 80/20) have the following features during processing of the soap bars and after washing with said bars:

Results of Standard Bar without Macadamia-based Additives/Esters of the Invention:

Runs well in the extruder
Good body with consistency, transparent or pearlescent effect
Good body texture.
When using the bar to wash hands with water, it gives a creamy foam, with large, but not compact bubbles.
After drying a soft feel was observed with no stickiness, and no gloss or shine observed either.
   Preparation of Soap Bars Comprising Macadamia-based Esters of the Invention

To the above soap bar containing 100% syndet base 96-143-1 and combo bar (25% syndet base 96-143-1 and 75% Bradford soap base 80/20) was added 1% Macadamia-based esters. The typical processing was followed, i.e, syndet base 96-143-1 or combo bar (25% syndet base 96-143-1 and 75% Bradford Tallow/coco soap base 80/20) was added to the amalgamator with esters, fragrance was incorporated, and the bar colored and refined as usual. The resulting bar was found to have the following additional beneficial properties in addition to those properties described above for the control bar:

Results of Soap Bars Comprising Macadamia-Based Esters of the Invention

Runs excellently in the extruder
Have excellent body with better consistency than the control bar,
Excellent transparent or pearlescent effect
Excellent body texture
When washed with water, the bar produces dense bubbles, and a slippery feel was observed when the bar was wet.
After drying, no stickiness was found on the skin.
It had a nice soft, silky feel.
Excellent shine and gloss was observed, and the skin felt well moisturized.

### EXAMPLE # 20

### Ref. No. 137-131

Transparent Combo Bar

Two transparent combo bars were prepared as follows:

Procedure:

1. Add ingredients from Glycerin to water in sequence, heat to 60° C. with mixing.
2. Add coco fatty acid and stearic acid and mix. Heat to 70° C. and allow to dissolve.
3. Add sodium stearate and soap base, mix, when it becomes clear add Tauranol I 78 C.
4. Mix and adjust moisture. A clear liquid is formed. Discharge.
   Observations:

Clarity and emolliency are better in Combo Bar A with Surfine AZI-A and Finsolv TN. Clarity is excellent in formulation B with Isostearyl Macadamiate, with good emolliency and feel. Clarity was tested against light.

### EXAMPLE # 21

### Ref. No. 137-51

Hand and Body Lotion with Sunscreen

Five formulations of hand and body lotion with sunscreen were prepared as follows:

| Ingredients/Tradename | | A | B | C | D | E | F |
|---|---|---|---|---|---|---|---|
| I. | Water | 75.7 | 75.7 | 75.7 | 75.7 | 75.7 | 75.7 |
| | Carbomer (Ultrez 10) | .15 | .15 | .15 | .15 | .15 | .15 |
| | Sorbitol 70% | 2 | 2 | 2 | 2 | 2 | 2 |
| | Tetrasodium EDTA | .2 | .2 | .2 | .2 | .2 | .2 |
| II. | Isostearyl Macadamiate | 6.5 | .2 | .2 | .2 | .2 | .2 |
| | PPG-3 Myristyl ether | — | 6.5 | — | — | — | — |
| | Macadamiate | | | | | | |
| | C12-15 Pareth-7 | — | — | 6.5 | — | — | — |
| | Macadamiate | | | | | | |
| | Ceteareth-4 Macadamiate | — | — | — | 6.5 | — | — |
| | Isopropyl Palmitate | — | — | — | — | 6.5 | — |
| | Isopropyl Myristate | — | — | — | — | — | 6.5 |
| | Cetyl alcohol | .7 | .7 | .7 | .7 | .7 | .7 |
| | Stearic acid | 1.5 | 1.5 | 1.5 | 1.5 | 1.5 | 1.5 |
| | Glyceryl Stearate SE | .8 | .8 | .8 | .8 | .8 | .8 |
| | Cetearyl alcohol & | 1 | 1 | 1 | 1 | 1 | 1 |
| | Ceteareth-20 | | | | | | |
| | Octylmetoxy cinnamate | 7 | 7 | 7 | 7 | 7 | 7 |
| | Octyl salicylate | 3 | 3 | 3 | 3 | 3 | 3 |
| | Benzophenone-3 | 3 | 3 | 3 | 3 | 3 | 3 |
| III. | Triethanolamine 99% | .9 | .9 | .9 | .9 | .9 | .9 |
| | Glydant | .2 | .2 | .2 | .2 | .2 | .2 |

Procedure:

1. Disperse Carbomer in water.

2. Heat to 70° C., add sorbitol and EDTA.

3. Add all phase II ingredients together and heat to 70° C.

4. Add phase II ingredients to phase I ingredients with good mixing.

5. Add triethanolamine, and continue mixing until 45° C.

6. Add Glydant, continue mixing to room temperature, discharge and label.

Results: Formulations A through F so prepared were tested for skin feel, emolliency, slip, stickiness, and moisturizing effect on a scale of 1 to 5, with 1 representing the best and 5 representing poor results. Results are as follows:

| | A | B | C | D | E | F |
|---|---|---|---|---|---|---|
| Skin feel | 2 | 2 | 3 | 3 | 4 | 4 |
| Emolliency | 2 | 1 | 3 | 2 | 4 | 5 |
| Slip | 2 | 2 | 3 | 3 | 3 | 4 |
| Stickiness | 2 | 2 | 3 | 3 | 4 | 4 |
| Moisturizing effect | 2 | 1 | 3 | 3 | 4 | 4 |

### EXAMPLE # 22

### Ref. No. 137-74

Waterproof Sunscreen Lotion

| Ingredients/Tradename | | A | B | C | D | E | F |
|---|---|---|---|---|---|---|---|
| I. | Deionized water | 72.75 | 72.75 | 72.75 | 72.75 | 72.75 | 72.75 |
| | Hydroxypropylcellulose | 10 | 10 | 10 | 10 | 10 | 10 |
| | (1% solution) | | | | | | |
| | Quaternium 15 | .15 | .15 | .15 | .15 | .15 | .15 |
| | Disodium EDTA | .05 | .05 | .05 | .05 | .05 | .05 |
| II. | Octylmethoxycinnamate | 7 | 7 | 7 | 7 | 7 | 7 |
| | octylsalicylate | 3 | 3 | 3 | 3 | 3 | 3 |
| | oxybenzone | 2 | 2 | 2 | 2 | 2 | 2 |
| | Isostearyl Macadamiate | 4 | — | — | — | — | — |
| | PPG-3 Myristyl | — | 4 | — | — | — | — |
| | Macadamiate | | | | | | |
| | C12-15 Pareth-7 | — | — | 4 | — | — | — |
| | Macadamiate | | | | | | |
| | Ceteareth-4 | — | — | — | 4 | — | — |
| | Macadamiate | | | | | | |
| | Isopropylmyristate | — | — | — | — | 4 | — |
| | Isopropylpalmitate | — | — | — | — | — | 4 |
| | Carbopol 954 | .25 | .25 | .25 | .25 | .25 | .25 |
| | Pemulen TR-1 | .2 | .2 | .2 | .2 | .2 | .2 |
| | Methylparaben | .05 | .05 | .05 | .05 | .05 | .05 |
| | Propylparaben | .15 | .15 | .15 | .15 | .15 | .15 |
| III. | Triethanolamine 99% | .4 | .4 | .4 | .4 | .4 | .4 |

Procedure:
1. Prepare Phase I by mixing until homogeneous.
2. Mix first four of Phase II ingredients in a separate vessel and stir until Oxybenzone is dissolved.
3. Disperse last four of Phase II ingredients with mixing.
4. Add Phase II ingredients to Phase I ingredients with agitation, continue mixing 40 minutes until smooth.
5. Add Phase III ingredient, stirring vigorously until smooth.
6. Discharge and label.

Formulations A through F so prepared were tested for skin feel, emolliency, slip stickiness, water rinseoff resistance and moisturizing effect on a scale of 1 to 5, with 1 representing the best and 5 representing poor results. Results were as follows:

| | A | B | C | D | E | F |
|---|---|---|---|---|---|---|
| Skin feel | 2 | 2 | 2 | 2 | 4 | 5 |
| Emolliency | 2 | 1 | 3 | 3 | 4 | 5 |
| Slip | 2 | 1 | 3 | 3 | 3 | 4 |
| Stickiness | 2 | 2 | 3 | 3 | 4 | 5 |
| Water rinseoff resistance | 2 | 1 | 3 | 2 | 3 | 4 |
| Moisturizing effect | 1 | 2 | 3 | 3 | 4 | 4 |

### EXAMPLE # 23

### Ref. No. 137-75

High SPF Sunscreen Lotion (Cold Process)

| Ingredients/Tradename | | A | B | C | D | E | F |
|---|---|---|---|---|---|---|---|
| I. | Abil WE-09 | 5 | 5 | 5 | 5 | 5 | 5 |
| | Cyclomethicone | 3 | 3 | 3 | 3 | 3 | 3 |
| | Isostearyl Macadamiate | 8 | — | — | — | — | — |
| | PPG-3 Myristyl | — | 8 | — | — | — | — |
| | Macadamiate | | | | | | |
| | C12-15 Pareth-.7 | — | — | 8 | — | — | — |
| | Macadamiate | | | | | | |
| | Ceteareth-4 Macadamiate | — | — | — | 8 | — | — |
| | Isopropyl Myristate | — | — | — | — | 8 | — |
| | Isopropyl Palmitate | — | — | — | — | — | 8 |
| | Abil Wax W9801 | 1 | 1 | 1 | 1 | 1 | 1 |
| | Octylmethoxycinnamate | 3 | 3 | 3 | 3 | 3 | 3 |
| | Octylsalicylate | 3 | 3 | 3 | 3 | 3 | 3 |
| | Benzophenone-3 | 2 | 2 | 2 | 2 | 2 | 2 |
| II. | Water | 68.4 | 68.4 | 68.4 | 68.4 | 68.4 | 68.4 |
| | Hydroxyethylcellulose | .8 | .8 | .8 | .8 | .8 | .8 |
| | (Natrosol l 250 HHR CS) | | | | | | |
| | Sodium Chloride | .8 | .8 | .8 | .8 | .8 | .8 |
| | Natrlfine TP-T | 5 | 5 | 5 | .8 | 5 | 5 |

Procedure:

1. Combine ingredients of Phase I and mix well.

2. Dissolve Hydroxyethylcellulose into vortex of the agitating water phase.

3. Allow the cellulose to fully hydrate prior to adding sodium chloride. Add Natrlfine TP-T. Mix until uniform.

4. Add ingredients of Phase II slowly into ingredients of Phase I with slow agitation.

5. Mix until homogeneous.

Formulations A through F so prepared were tested for skin feel, emolliency, slip, water rinseoff resistance and moisturizing effect on a scale of 1 to 5, with 1 representing the best and 5 representing poor results. Results were as follows:

| | A | B | C | D | E | F |
|---|---|---|---|---|---|---|
| Skin feel | 2 | 1 | 3 | 3 | 4 | 4 |
| Emolliency | 2 | 1 | 3 | 3 | 5 | 5 |
| Slip | 2 | 2 | 2 | 3 | 4 | 4 |
| Water rinseoff resistance | 1 | 1 | 3 | 2 | 5 | 5 |
| Moisturizing effect | 2 | 2 | 2 | 3 | 4 | 5 |

### EXAMPLE #24

### Ref. No. 137-91

High SPF Sunscreen Lotion

| Ingredients/Tradename | | A | B | C | D | E | F |
|---|---|---|---|---|---|---|---|
| I. | Abil WE-09 | 5 | 5 | 5 | 5 | 5 | 5 |
| | Cyclomethicone | 3 | 3 | 3 | 3 | 3 | 3 |
| | Isostearyl Macadamiate | 8 | — | — | — | — | — |
| | PPG-3 Myristyl | — | 8 | — | — | — | — |
| | Macadamiate | | | | | | |
| | C12-15 Pareth-7 | — | — | 8 | — | — | — |
| | Macadamiate | | | | | | |
| | Ceteareth-4 Macadamiate | — | — | — | 8 | — | — |
| | Isopropyl Myristate | — | — | — | — | 8 | — |
| | Isopropyl Palmitate | — | — | — | — | — | 8 |
| | Abil Wax W9801 | 1 | 1 | 1 | 1 | 1 | 1 |
| | Octylmethoxycinnamate | 3 | 3 | 3 | 3 | 3 | 3 |
| | Octylsalicylate | 3 | 3 | 3 | 3 | 3 | 3 |
| | Benzophenone-3 | 2 | 2 | 2 | 2 | 2 | 2 |
| II. | Water | 68.4 | 68.4 | 68.4 | 68.4 | 68.4 | 68.4 |
| | Hydroxyethylcellulose | .8 | .8 | .8 | .8 | .8 | .8 |
| | (Natrosol 250 HHR CS) | | | | | | |
| | Sodium chloride | .8 | .8 | .8 | .8 | .8 | .8 |
| | Natrifine 137 T | 5 | 5 | 5 | 5 | 5 | 5 |

Procedure:
1. Combine ingredients in Phase I and mix well.
2. Dissolve Hydroxyethylcellulose into vortex of the agitating water phase. Allow the cellulose to fully hydrate prior to adding the sodium chloride. Add Natrlfine 137-T. Mix until uniform.
3. Add ingredients of Phase II slowly into ingredients of Phase I with slow agitation.
4. Mix until homogeneous.

Formulations A through F so prepared were tested for skin feel, emolliency, slip, water rinseoff resistance and moisturizing effect on a scale of 1 to 5, with 1 representing the best and 5 representing poor results. Results were as follows:

| | A | B | C | D | E | F |
|---|---|---|---|---|---|---|
| Skin feel | 2 | 2 | 2 | 3 | 4 | 4 |
| Emolliency | 2 | 1 | 2 | 2 | 5 | 5 |
| Slip | 2 | 2 | 3 | 3 | 4 | 5 |
| Water rinseoff resistance | 1 | 1 | 2 | 3 | 5 | 5 |
| Moisturizing effect | 2 | 1 | 2 | 3 | 4 | 4 |

### EXAMPLE #25

### Ref. No. 137-78

Sunblock Cream

| Ingredients/Tradename | | A | B | C | D | E | F |
|---|---|---|---|---|---|---|---|
| I. | Water | 67.7 | 67.7 | 67.7 | 67.7 | 67.7 | 67.7 |
| | Magnesium Aluminum | 1.5 | 1.5 | 1.5 | 1.5 | 1.5 | 1.5 |
| | Silicate | | | | | | |
| | Propylene glycol | 3 | 3 | 3 | 3 | 3 | 3 |
| | Triethanolamine | .6 | .6 | .6 | .6 | .6 | .6 |
| II. | Benzophenone-3 | 5 | 5 | 5 | 5 | 5 | 5 |
| | Isostearyl Macadamiate | 7.5 | — | — | — | — | — |
| | PPG-3 Myristyl | — | 7.5 | — | — | — | — |
| | Macadamiate | | | | | | |
| | C12-15 Pareth-7 | — | — | 7.5 | — | — | — |
| | Macadamiate | | | | | | |
| | Ceteareth-4 Macadamiate | — | — | — | 7.5 | — | — |
| | Isopropyl Myristate | — | — | — | — | 7.5 | — |
| | Isopropyl Palmitate | — | — | — | — | — | 7.5 |
| | Mineral oil | 4 | 4 | 4 | 4 | 4 | 4 |
| | Stearic acid | 2.2 | 2.2 | 2.2 | 2.2 | 2.2 | 2.2 |
| | Glyceryl stearate | .5 | .5 | .5 | .5 | .5 | .5 |
| | Cetyl alcohol | .5 | .5 | .5 | .5 | .5 | .5 |
| | Octylmethoxycinnamate | 7.5 | 7.5 | 7.5 | 7.5 | 7.5 | 7.5 |

Procedure:

1. Heat water to 80° C.

2. Add magnesium aluminium silicate slowly with stirring, mix until smooth.

3. Add remaining ingredients of Phase I and mix until smooth while maintaining temperature.

4. Heat ingredients of Phase II to 75° C., and add to ingredients of Phase I.

5. Mix until cool to 45° C. and discharge.

Formulations A through F so prepared were tested for skin feel, emolliency, slip, stickiness, moisturizing effect and water rinseoff resistance on a scale of 1 to 5, with 1 representing the best and 5 representing poor results. Results were as follows:

| | A | B | C | D | E | F |
|---|---|---|---|---|---|---|
| Skin feel | 2 | 1 | 3 | 3 | 5 | 4 |
| Emolliency | 2 | 2 | 2 | 3 | 4 | 4 |
| Slip | 2 | 1 | 2 | 3 | 5 | 5 |
| Stickiness | 2 | 2 | 2 | 2 | 5 | 4 |
| Moisturizing effect | 1 | 2 | 2 | 2 | 4 | 5 |
| Water rinseoff resistance | 2 | 1 | 3 | 2 | 4 | 4 |

### EXAMPLE #26

### Ref. No. 137-61

Non-Whitening Antiperspirant Stick

| Ingredients/Tradename | | A | B | C | D | E | F |
|---|---|---|---|---|---|---|---|
| I. | Cyclomethicone | 39 | 39 | 39 | 39 | 39 | 39 |
| | Stearyl Alcohol | 18 | 18 | 18 | 18 | 18 | 18 |
| | Hydrogeneted castor oil | 5 | 5 | 5 | 5 | 5 | 5 |
| | Isostearyl Macadamiate | 5 | — | — | — | — | — |
| | PPG-3Myristyl Macadamiate | — | 5 | — | — | — | — |
| | C12-15 Pareth-7 Macadamiate | — | — | 5 | — | — | — |
| | Ceteareth-4 Macadamiate | — | — | — | 5 | — | — |
| | Isopropyl Myristate | — | — | — | — | 5 | — |
| | Isopropyl palmitate | — | — | — | — | — | 5 |
| | Finsolv116 | 10 | 10 | 10 | 10 | 10 | 10 |
| II. | Aluminum Zirconium | 20 | 20 | 20 | 20 | 20 | 20 |
| | tetrachlorohydroxy gly | | | | | | |
| | Talc | 2 | 2 | 2 | 2 | 2 | 2 |
| | silica | 1 | 1 | 1 | 1 | 1 | 1 |

Procedure:
1. Combine ingredients of Phase I and heat to 75° C. with mixing.
2. Add Phase I powders. Mix 15 minutes or until completely dispersed. Maintain temperature of 75° C.
3. Cool to 55° C., and mold sticks.

Formulations A through F so prepared were tested for emolliency, skin feel, slip and stick structure on a scale of 1 to 5, with 1 representing the best and 5 representing poor results. Results are as follows:

| | A | B | C | D | E | F |
|---|---|---|---|---|---|---|
| Emolliency | 2 | 1 | 3 | 3 | 4 | 4 |
| Skin feel | 2 | 1 | 2 | 2 | 5 | 5 |
| Slip | 2 | 2 | 2 | 3 | 4 | 4 |
| Stick structure | 2 | 1 | 2 | 3 | 5 | 5 |

### EXAMPLE #27

### Ref. No. 137-60

Deodorant Stick

| Ingredients/Tradename | A | B | C | D | E | F |
|---|---|---|---|---|---|---|
| Propylene glycol | 71 | 71 | 71 | 71 | 71 | 71 |
| Water | 14 | 14 | 14 | 71 | 71 | 71 |
| Isostearyl Macadamiate | 3 | — | — | — | — | — |
| PPG-3 Myristyl Macadamiate | — | 3 | — | — | — | — |
| C12-15 Pareth-7 | — | — | 3 | — | — | — |
| Macadamiate | | | | | | |
| Ceteareth-4 Macadamiate | — | — | — | 3 | — | — |
| Isopropyl Myristate | — | — | — | — | 3 | — |
| Isopropylpalmitate | — | — | — | — | — | 3 |
| Sodium Stearate | 5 | 5 | 5 | 5 | 5 | 5 |
| Triclosan | .2 | .2 | .2 | .2 | .2 | .2 |
| Pentadoxynol-200 | 6.6 | 6.5 | 6.5 | 6.5 | 6.5 | 6.5 |
| Triethanolamine 99% | .3 | .3 | .3 | .3 | .3 | .3 |

Procedure:

1. Charge all ingredients into a suitable vessel.

2. Heat to 80° C., with mixing, allowing all ingredients to dissolve.

3. Cool to 60° C. and cast into stick moulds.

Formulations A through F so prepared were tested for skin feel, slip, stick structure, emolliency and tackiness on a scale of 1 to 5, with 1 representing the best and 5 representing poor results. Results were as follows:

| | A | B | C | D | E | F |
|---|---|---|---|---|---|---|
| Skin feel | 2 | 1 | 3 | 3 | 5 | 4 |
| Slip | 2 | 1 | 3 | 3 | 4 | 4 |
| Stick structure | 1 | 2 | 3 | 2 | 5 | 5 |
| Emolliency | 2 | 1 | 2 | 3 | 4 | 4 |
| Tackiness | 2 | 2 | 3 | 3 | 4 | 5 |

### EXAMPLE #28

### Ref. No. 137-52

Emollient Cleansing Lotion

| Ingredients/Tradename | | A | B | C | D | E | F |
|---|---|---|---|---|---|---|---|
| I. | Water | 80.4 | 80.4 | 80.4 | 80.4 | 80.4 | 80.4 |
| | Fizul MD 318 C | 1 | 1 | 1 | 1 | 1 | 1 |
| | Glycerin | 3 | 3 | 3 | 3 | 3 | 3 |
| | Carbopol ETD 2001 | .2 | .2 | .2 | .2 | .2 | .2 |
| | Resin | | | | | | |
| | Tetrasodium EDTA | .1 | .1 | .1 | .1 | .1 | .1 |
| II. | Isostearyl Macadamiate | 6.5 | — | — | — | — | — |
| | PPG-3 Myristyl | — | 6.5 | — | — | — | — |
| | Macadamiate | | | | | | |
| | C12-15 Pareth-7 | — | — | 6.5 | — | — | — |
| | Macadamiate | | | | | | |
| | Ceteareth-4 Macadamiate | — | — | — | 6.5 | — | — |
| | Isopropyl Myristate | — | — | — | — | 6.5 | — |
| | Isopropyl Palmitate | — | — | — | — | — | 6.5 |
| | Caprylic/capric | 1 | 1 | 1 | 1 | 1 | 1 |
| | Triglycerides | | | | | | |
| | Stearic acid | 3 | 3 | 3 | 3 | 3 | 3 |
| | Glyceryl stearate SE | 2 | 2 | 2 | 2 | 2 | 2 |
| III. | Water | 1 | 1 | 1 | 1 | 1 | 1 |
| | Triethanolamine 99% | .8 | .8 | .8 | .8 | .8 | .8 |
| IV | Germaben | 1 | 1 | 1 | 1 | 1 | 1 |

Procedure:

1. Disperse Carbomer into water.

2. Add balance of Phase I ingredients and heat to 75° C.

3. Mix Phase II ingredients together and heat to 75° C.

4. Add Phase II ingredients to Phase I ingredients, with mixing.

5. Combine Phase III ingredients, then add to the above and begin cooling.

6. At 40° C., add Phase IV ingredients and continue cooling to 30° C.

Formulations A through F so prepared were tested for slip, skin feel, and emolliency on a scale of 1 to 5, with 1 represent the best and 5 representing poor. Results are as follows:

| | A | B | C | D | E | F |
|---|---|---|---|---|---|---|
| Skin feel | 2 | 1 | 3 | 3 | 5 | 4 |
| Slip | 1 | 1 | 3 | 3 | 4 | 4 |
| Emolliency | 2 | 1 | 3 | 2 | 4 | 5 |

### EXAMPLE # 29

### Ref. No. 137-51

Hand and Body Lotion

| Ingredients/Tradename | | A | B | C | D | E | F |
|---|---|---|---|---|---|---|---|
| I. | Water | 85.75 | 85.75 | 85.75 | 85.75 | 85.75 | 85.75 |
| | Carbomer (Ultrez 10) | .15 | .15 | .15 | .15 | .15 | .15 |
| | Sorbitol 75% | 2 | 2 | 2 | 2 | 2 | 2 |
| | Tetrasodium EDTA | .2 | .2 | .2 | .2 | .2 | .2 |
| II. | Isostearyl Macadamiate | 6.5 | — | — | — | — | — |
| | PPG-3 Myristyl | — | 6.5 | — | — | — | — |
| | Macadamiate | | | | | | |
| | C12-15 Pareth-7 | — | — | 6.5 | — | — | — |
| | Macadamiate | | | | | | |
| | Ceteareth-4 Macadamiate | — | — | — | 6.5 | — | — |
| | Isopropyl Myristate | — | — | — | — | 6.5 | — |
| | Isopropyl Palmitate | — | — | — | — | — | 6.5 |
| | Cetyl alcohol | .7 | .7 | .7 | .7 | .7 | .7 |
| | Stearic acid | 1.5 | 1.5 | 1.5 | 1.5 | 1.5 | 1.5 |
| | Glyceryl stearate SE | .8 | .8 | .8 | .8 | .8 | .8 |
| | Cetryl alcohol & | 1 | 1 | 1 | 1 | 1 | 1 |
| | ceteareth 20 | | | | | | |
| III. | Triethanolamine 99% | .9 | .9 | .9 | .9 | .9 | .9 |
| | Germaben II | .3 | .3 | .3 | .3 | .3 | .3 |

Procedure:

1. Disperse carbomer in water.

2. Heat to 70° C.

3. Add sorbitol 70% and tetrasodium EDTA.

4. Weigh all Phase II ingredients together and heat to 70° C.

5. Add ingredients of Phase II to ingredients of Phase I with mixing.

6. Add triethanolamine, continue mixing until 45° C.

7. Add preservative.

Formulations A through F so prepared were tested for skin feel, slip and emolliency on a scale of 1 to 5, with 1 representing the best and 5 representing poor results. Results were as follows:

| | A | B | C | D | E | F |
|---|---|---|---|---|---|---|
| Skin feel | 2 | 1 | 2 | 3 | 4 | 5 |
| Slip | 2 | 1 | 2 | 2 | 5 | 4 |
| Emolliency | 1 | 2 | 3 | 3 | 5 | 4 |

### EXAMPLE # 30

### Ref. No. 137-41

### Foam Test

Foaming Characteristics of Macadamia oil-based Surfactants

Test comprises 0.1 gram macadamia oil-based surfactant in 100 ml. water in a 500 ml. graduated cylinder with stopper. Give 10 strokes on the cylinder by hand. The results were measured in terms of foam height, initially and after 5 minutes, the nature of the foam, and the durability of the foam.

### EXAMPLE #30-A

| | Betaine of | |
|---|---|---|
| | Macadamia Oil | Cocobetaine |
| | (Example #4) | (Control/Comparison) |
| Initial height (0 minutes) | 170 ml. | 150 ml. |
| Final height (5 minutes) | 150 ml. | 100 ml. |
| Durability of foam | stable | stable |
| Nature of foam | uniform thick rich | mixture of thick |
| | dense foam/bubbles | and thin foam |

### EXAMPLE #30-B

| | Diethyl Sulphate | FINQUAT- | FINQUAT |
|---|---|---|---|
| | Quaternary of | CT | CT (P) |
| | Macadamia Oil | (control/ | (control/ |
| | (Example #5) | comparison) | comparison) |
| Initial height (0 minutes) | 220 ml. | 120 ml. | 105 ml. |
| Final height (5 minutes) | 200 ml. | 100 ml. | 100 ml. |
| Durability of foam | stable | scattered | scattered |
| Nature of foam | uniform thick, | thin foam, | no foam |
| | dense, rich | disappears | small |
| | foam/bubbles | quickly | bubbles |

EXAMPLE 30-C

| | Macadamia Oil MEA, | TAURANOL |
|---|---|---|
| | Tauranol WS | WS CONS |
| | CONS and Water | (Control/ |
| | (Example #2) | Comparison) |
| Initial height (0 minutes) | 200 ml. | 200 ml. |
| Final height (5 minutes) | 170 ml. | 150 ml. |
| Durability of foam | stable | stable |
| Nature of foam | unifom thick | large bubbles |
| | dense rich | scattered/thin |
| | foam/bubbles | |

### EXAMPLE # 31

### Ref. No.137-46

Conditioning Shampoo

This is a comparison of Macadamia Betaine (Example # 4) to Cocoamidopropylbetaine (CAPB), a commercially available betaine from McIntyre Group, University Park, Ill. Table IV below sets forth the ingredients and the test results follow.

TABLE IV

| Ingredients/Tradename | A | B |
|---|---|---|
| Water | 55.7 | 55.7 |
| Citric acid | 0.175 | 0.175 |
| Sodium Citrate | 0.500 | 0.500 |
| Versene Na2 Crystal | 0.050 | 0.050 |
| Hydrosoy 2000SF | 0.100 | 0.100 |
| Standapol A | 36.00 | 36.00 |
| Mackam L | 5.400 | — |
| Macadamia Betaine | — | 5.400 |
| Mackamide LLM | 0.500 | 0.500 |
| Fragrance 808999 | 0.300 | 0.300 |
| Kathon CG | 0.025 | 0.025 |
| Sodium Chloride | 1.250 | 1.250 |
| Total | 100 | 100 |

Procedure:

1. Charge water and heat to 70° C.

2. Add other ingredients in the order listed above.

3. Bring temperature to 75° C.

4. Mix well until uniform.

5. Cool to 45° C.

6. Add fragrance and preservative.

7. Add Sodium Chloride.

8. Mix gently, cooling to 35° C.

The properties of formulations A and B are as follows:

| | Formulation A | Formulation B |
|---|---|---|
| Appearance | Clear gel | Clear gel |
| PH, as is | 5.0 | 5.02 |
| Viscosity (cps) | 1150 | 1600 |

Both Formulations A and B are clear, viscous gels, but Formulation B is more viscous. Formulations A and B so prepared were tested for skin feel, emolliency, and slip on a scale of 1 to 5, with 1 representing the best and 5 representing poor results. The results were as follows:

| | Formulation A | Formulation B |
|---|---|---|
| Skin Feel (scalp) | 3 | 1 |
| Emolliency (scalp) | 3 | 2 |
| Slip (smoothness) | 3 | 1 |

Formulation B, the product of invention, gave superior skin feel, emolliency, and slip.

### EXAMPLE #32

### Ref. No. 137-44

Shower Gel

This is a comparison of Macadamia Betaine (Example # 4) to Cocoamidopropylbetaine (CAPB), a commercially available betaine from McIntyre Group. Table V below sets forth the ingredients and the test results follow.

TABLE V

| Ingredients/Tradename | | A | B |
|---|---|---|---|
| A. | Water | 20.800 | 20.800 |
| | Varsene 100 XL | 0.050 | 0.050 |
| | Standapol ES-2 | 20.00 | 20.00 |
| | Hamposyl L-30 | 20.00 | 20.00 |
| | Crothix | 0.200 | 0.200 |
| | Glycerox HE | 3.00 | 3.00 |
| | Germaben II | 1.00 | 1.00 |
| | Mackam L | 30.00 | — |
| | Macadamia Betaine Ex. 4 | — | 30.00 |
| B. | Mackamide LLM | 3.00 | 3.00 |
| | Citric acid, 50% Solution | 0.45 | 0.45 |
| | Fragrance #154841-06 | 1.500 | 1.500 |
| | Total | 100 | 100 |

Procedure:
1. Charge ingredients of part A and heat to 70° C.
2. Bring temperature to 75° C.
3. Mix well until uniform.
4. Add ingredients of part B and mix well, avoiding aeration.
5. Cool to 45° C. with gentle mixing.
6. Add ingredients of part C.
7. Mix well (gently).
8. Allow to deaerate by standing at 45° C.
9. Cool to 35° C.

The properties of the two formulations A and B are as follows:

| | Formulation A | Formulation B |
|---|---|---|
| Appearance | Clear gel | Clear gel |
| PH, as is | 5.6 | 5.8 |
| Viscosity (cps) | 1400 | 2600 |

Both Formulations A and B are clear, viscous gels, but Formulation B is more viscous. Formulations A and B so prepared were tested for skin feel, emolliency, and slip on a scale of 1 to 5, with 1 representing the best and 5 representing poor results. The results were as follows:

| | Formulation A | Formulation B |
|---|---|---|
| Skin Feel (scalp) | 4 | 1 |
| Emolliency (scalp) | 4 | 2 |
| Slip (smoothness) | 4 | 1 |

Formulation B, the product of invention, gave superior skin feel, emolliency, and slip.

### EXAMPLE #33

### Ref. No. 137-45

Clear and Mild Skin Cleansing Gel

This is a comparison of Macadamia Betaine vs CAPB when used in combination with Example # 2. Table VI below sets forth the ingredients and test results follow.

| | Reference Nos. | | | |
|---|---|---|---|---|
| Ingredients/Tradenames | 137-45 A | 137-45 B | 137-45 C | 137-45 D |
| Water | 50.5 | 50.5 | 50.5 | 50.5 |
| Standapol EA-2 | 15 | 15 | 15 | 15 |
| Tauranol I-78C | 6 | 6 | 6 | 6 |
| Cocobetaine | 10 | — | 10 | — |
| Macadamia Betaine | — | 10 | — | 10 |
| Macadamiaamide- | 15 | 15 | — | — |
| Taurate Blend (Ex. # 2) | | | | |
| Tauranol WS CONC | — | — | 15 | 15 |
| PEG-150 Pentaerythrityl | 2 | 2 | 2 | 2 |
| Stearate | | | | |
| Germaben II | .5 | .5 | .5 | .5 |
| Baby Fragrance | 1 | 1 | 1 | 1 |
| Total | 100 | 100 | 100 | 100 |

Procedure:
1. Charge water and heat to 70° C.
2. Add other ingredients in the order listed above.
3. Bring temperature to 75° C.
4. Mix well until uniform.
5. Cool to 45° C.
6. Add fragrance.
7. Add Germaben II and mix.
8. Cool to 35° C.

The properties of Formulations 1-4 of Example #33 are as follows:

| | 1 | 2 | 3 | 4 |
|---|---|---|---|---|
| Appearance | Clear Gel | Clear Gel | Clear Gel | Clear Gel |
| PH, as is | 6.2 | 6.3 | 5.9 | 6.1 |
| Viscosity (cps) | 20000 | 28000 | 31000 | 29000 |

Formulations 1-4 so prepared were tested for skin feel, emolliency, and slip on a scale of 1 to 5, with 1 representing the best and 5 representing poor result. The results were as follows:

| | 1 | 2 | 3 | 4 |
|---|---|---|---|---|
| Skin feel | 4 | 1 | 3 | 1 |
| Emolliency | 3 | 1 | 4 | 1 |
| Slip | 4 | 2 | 3 | 2 |

### EXAMPLE # 34

### Ref. No. 137-43

Gentle Facial Cleaner

This is a comparison of Macadamia betaine (Example #4) to Disodium Cocoamphodiacetate, a commercially available amphoteric betaine from McIntyre Group

TABLE VII

| Ingredients/Tradenames | A | B |
|---|---|---|
| 1. Water | 91.39 | 91.39 |
| 2. Macadamiabetaine (Example #4) | — | 8.00 |
| 3. Pluracare L-64 | 2.00 | — |
| 4. Makam 2C | 6.00 | — |
| 5. Citric acid | .01 | .01 |
| 6. Ajidew N-50 | .10 | .10 |
| 7. Germall Plus | .2 | .2 |
| 8. Ritapan DL | .1 | .1 |
| 9. Biopol HC | .2 | .2 |
| Total | 100 | 100 |

Procedure:

1. Charge water (1) and heat to 70 C

2. Add other ingredients (2) through (6) in the order listed above.

3. Bring temperature to 75° C.

4. Mix well until uniform.

5. Cool to 45° C.

6. Add ingredients (7) through (9) in the order set forth above.

7. Mix gently, cooling to 35° C.

The properties of the two formulations A and B of Example #34 are as follows:

| | Formulation A | Formulation B |
|---|---|---|
| Appearance | Clear liquid | Clear liquid |
| PH, as is | 8.5 | 7.02 |
| Viscosity (cps) | Very thin liquid | 3600 |

Both Formulations A and B are clear, viscous gels, but Formulation B is more viscous. Formulations A and B so prepared were tested for skin feel, emolliency, and slip on a scale of 1 to 5, with 1 representing the best and 5 representing poor results. The results were as follows:

| | Formulation A | Formulation B |
|---|---|---|
| Skin feel (scalp) | 4 | 1 |
| Emolliency (scalp) | 4 | 2 |
| Slip (smoothness) | 4 | 1 |

Formulation B, the product of invention, gave superior skin feel, emolliency, and slip.

### EXAMPLE # 35

### Ref. No.137-47

Shower and Bath Gel

This is a comparison of Macadamia-based betaine and Macadamia-based ester emollient to commercial cocobetaine and commercial ester emollients. Table VIII below sets forth the ingredients and test results follow.

TABLE VIII

| | | Reference Nos. | | | |
|---|---|---|---|---|---|
| Ingredients/Tradenames | | 137-47 A | 137-47 B | 137-47 C | 137-47 D |
| I. | Water | 30.5 | 30.5 | 30.5 | 30.5 |
| | Standapol ES-40 | 30 | 30 | 30 | 30 |
| | Tauranol I-78C | 5 | 5 | 5 | 5 |
| | Cocobetaine | 10 | — | 10 | — |
| | Macadamia Betaine | — | 10 | — | 10 |
| | Macadamiaamide | 20 | 20 | 20 | 20 |
| | MEA Taurate | | | | |
| | Ex. #2 | | | | |
| II. | Finsolv TN | 3 | — | 3 | — |
| | PPG-3 Myristyl | — | 3 | — | 3 |
| | Macadamiate | | | | |
| III. | Germaben II | .5 | .5 | .5 | .5 |
| | Baby Fragrance | 1 | 1 | 1 | 1 |
| | Total | 100 | 100 | 100 | 100 |

Procedure:
1. Charge water and heat to 70° C.
2. Add other ingredients in the order listed above.
3. Bring temperature to 75° C.
4. Mix well until uniform.
5. Cool to 45° C.
6. Add fragrance.
7 Add Germaben II. Mix.
8. Cool to 35° C.

The properties of the formulations 1-4 of Example # 35 are as follows:

| | 1 | 2 | 3 | 4 |
|---|---|---|---|---|
| Appearance | Thin liquid | Thin liquid | Viscous liquid | Viscous |
| | | | | liquid |
| PH, as is | 6.2 | 6.3 | 5.9 | 6.1 |
| Viscosity (cps) | — | — | 9000 | 8000 |

### EXAMPLE # 36

### Ref. No. 137-42

2 in 1 Clear Conditioning Shampoo

This is a comparison of Macadamiaamide MEA, Diethyl sulfate quatemium of Macadamia with commercially available substantive quaternary ammonium compounds. Table IX below sets forth the ingredients and test results follow.

TABLE IX

| Ingredients/Tradenames | A | B | C | D |
|---|---|---|---|---|
| Water | 42.8 | 42.8 | 42.8 | 42.8 |
| Standapol ES-40 | 30 | 30 | 30 | 30 |
| Macadamiaamide (Example #2) | 20 | 20 | — | — |
| Tauranol WS CONS | — | — | 20 | 20 |
| QUAT of Macadamia (Example #6) | 5 | — | — | — |
| Finquat CT | — | 5 | 5 | — |
| Finquat CT-P | — | — | — | 5 |
| PEG-150 Pentaerythrityl Stearate | 1 | 1 | 1 | 1 |
| DMDM Hydantoin | .2 | .2 | .2 | .2 |
| Baby Fragrance | 1 | 1 | 1 | 1 |
| Citric acid 25% for pH 6 | Q.S | Q.S | Q.S | Q.S |
| Total | 100 | 100 | 100 | 100 |

Procedure:
1. Charge water and heat to 70° C.
2. Add other ingredients in the order listed above.
3. Bring temperature to 75° C.
4. Mix well until uniform.
5. Cool to 45° C.
6. Add fragrance.
7. Adjust pH to 6.0.
8. Package

The properties of the formulations A-D of Example #36 as follows:

| | A | B | C | D |
|---|---|---|---|---|
| Appearance | Clear | Clear Gel | Thin viscous liquid | Thin viscous |
| | Gel | | | liquid |
| PH, as is | 6.2 | 6.3 | 5.9 | 6.1 |
| Viscosity (cps) | 9000 | 7000 | 3000 | 2000 |

Formulations A and B gave superior skin feel, emolliency, and slip.

### EXAMPLE # 37

### Ref. No. 137-66

Comparative Substantivity Properties

Sustantivity is defined as the ability of the quaternary (or Cationic) substance to be attracted to an anionic surface such as hair and wool. Substantivity is taken as a conditioning efficacy parameter for hair care products. In the industry, the Rubine Dye Uptake Test is typically performed to screen for the property of substantivity. A contol fabric swatch of wool is treated in water without any treatment with a quaternary compound. The test articles, i.e., fabric swatches, are applied with a certain level of activity to the same weight fabric swatches as the control swatch. The treated fabric swatches are rinsed and then further treated in a solution of anionic acid dye such as Rubin Red Dye. The treatment with dye solution allows the fabric to attract the strong anionic dye solution with the help of a quaternary compound that has been applied to the swatch. The depth of color is stronger or weaker depending upon the substantivity (higher or lower) of the quaternary. The deeper the color, the greater the substantivity of the quaternary.

The Rubine Dye Uptake Test was performed using the following materials:
1. Wool (Worsted) swatches:
   Size: 3.5 inches×5.5 inches
   Weight: 2.6 gm
2. Dye solution:
   0.50 gms. Dye (Direct Red # 207, i.e., Lumicrease Bordeaux 3LR, Clariant, Inc., Charlotte, N.C.)
   0.125 gms. Glacial Acetic Acid
   q.s. Water
   1000 mls.
3. Test Sample Quaternary Solution:
   0.5% Active Quaternary in Water, i.e., 1.0 gm. of Active Quaternary in 200 ml. Water.
   Procedure:

The control swatch is treated in water without quaternary compound. Separate swatches for each quaternary substance are treated for 5 minutes with mild stirring in separate beakers. The swatches are then individually (separately) rinsed with water.

The control swatch and treated swatches are further treated (each one in a separate beaker) with dye solution for 5 minutes and rinsed in tap water at 40 C. The swatches are allowed to air dry. The depth of the dye uptake is compared. The higher the color depth, the higher the substantivity of the quaternary.

The results of the test are set forth in Table X below, where a substantivity rating of 1 is the best, and a rating of 10 is poor.

TABLE X

| | % Active | | |
|---|---|---|---|
| Test Articles | Quaternary | INCI Name | Ratings |
| No Conditioner (control) | None | — | 10 |
| FINQUAT-CT | 0.50 | Quaternium 75 | 3 |
| Invention Example# 5 | 0.50 | DES Quat of | 1 |
| | | Macadamia | |
| Stearalkonium Chloride | 0.50 | Stearalkonium | 2 |
| | | Chloride | |
| Cetrimonium Chloride | 0.5 | Cetrimonium Chloride | 2 |
| Poly Quaternium 7 | 0.5 | Poly Quaternium 7 | 2 |

As can be seen in Table X, DES quaternary of Macadamia (Example # 5) shows superior substantivity in this group of quaternaries tested. This is indicative of high substantivity for its use as a conditioning agent for hair care products. Hair is similar to wool fibers. Accordingly, wool is used as a sample textile material, serving a dual purpose in testing quaternaries.

### EXAMPLE #38

### Ref. No. 137-65

Comparative Conditioning Properties

Conditioning efficacy is judged by evaluating wet and dry comb characteristics and flyaway features of the hair tresses treated with the conditioners. A comparative study was done using the quaternary of this invention (Product of Example #5) as compared to other commercially marketed products.

A prototype formulation of Conditioner Base was made as shown below:

| | | |
|---|---|---|
| Phase A | | |
| Water: | Q.S to 100 | parts |
| Hydroxyethyl Cellulose: | 0.40 | Parts |
| Propylene Glycol: | 10.00 | Parts |
| PEG-75 Lanolin: | 0.25 | Parts |
| Phase B | | |

| | |
|---|---|
| Conditioners: | Q.S for |
| | 2.5% Active |

Procedure:

Mix hydroxyethylcellulose (i.e., Natrosol 250HHRCS) in water. Dissolve completely at 50° C. Add propylene glycol and PEG 75 Lanolin. Mix for 30 minutes, clear system after adding three ingredients in water. Add Conditioners shown in the Table XI. Rinse hair tresses in tap water for 1 second. Transfer rinsed hair tresses into combined portion of phases A & B. Mix for 5 minutes. Remove tresses and rinse with tap water running on it for 15 seconds. Hang each wet sample by clamping one end of the tresses. Perform a wet and dry comb test. The attached table gives the results of comparative conditioning properties, with a rating of 1 being the best, and a rating of 10 being poor.

TABLE XI

| Conditioners | Wet Comb | Dry Comb | Flyaway (Inches) |
|---|---|---|---|
| Contol (No conditioner) | 10 | 10 | 3 |
| Finquat CT | 6 | 2 | 2.5 |
| Macadamia quat (Ex.# 5) | 1 | 1 | 1 |
| Stearylalkonium Chloride | 5 | 5 | 4.5 |
| Cetrimonium Chloride | 7 | 3 | 4 |
| Polyquat 7 | 7 | 4 | 4 |

As can be seen in Table XI Macadamia Quat (the product of invention Example #5) shows superior conditioning properties in this group of quaternaries tested. This is indicative of its benefits for use as a conditioning agent for hair care products.

TABLE XII
Identification of Trade or Generic Listed Preparation Ingredients

| Trade/Generic Material | Identification | Source |
|---|---|---|
| Finquat CT-P (CONS) | Quaternium 89 | Finetex Inc., NJ |
| Finquat CT | Quaternium 75 | Finetex Inc., NJ |
| Aminol HCA | Cocoamide | Finetex Inc., NJ |
| Finsolv SLB-101 | Dimethicone PEG/PPG-20/23 Benzoate | Finetex Inc., NJ |
| Finsolv SLB-201 | Dimethicone PEG-8 Benzoate | Finetex Inc., NJ |
| Finsolv TN | C12-C15 Alkyl Benzoate | Finetex Inc., NJ |
| Finester EH 25 | C12-C15 Alkyl Octanoate | Finetex Inc., NJ |
| Finsolv PL-62 | Poloxamer 182 Dibenzoate | Finetex Inc., NJ |
| Finester DOM-R | Dioctyl Maleate | Finetex Inc., NJ |
| Finsolv EMG-20 | Methyl Gluceth-20 Benzoate | Finetex Inc., NJ |
| Finsolv PL-355 | Poloxamer 105 Benzoate | Finetex Inc., NJ |
| Finsolv BCR-111 | Cetyl Ricinoleate Benzoate | Finetex Inc., NJ |
| Finsolv BCO-115 | Castor Oil Benzoate | Finetex Inc., NJ |
| Finsolv BOHS-111 | Ethylhexyl Hydroxystearate Benzoate | Finetex Inc., NJ |
| Tauranol I78C | Sodium Cocyl Isethionate | Finetex Inc., NJ |
| Tauranol WS (conc) | Sodium Methyl Cocyl Taurate | Finetex Inc., NJ |
| Surfine-AZI-A | Nonoxynol-10 Carboxylate | Finetex Inc., NJ |
| Natrlfine 137-T | Behenyl Benhenate, Titanium Dioxide | Finetex Inc., NJ |
| Solulan 16 | Laneth-16, Ceteth 16, oleth-16 & Stearate | Amerchol Edison, NJ |
| Drakeol 9 | Light Mineral Oil | Panorco, PA |
| Hystrene 9718 | Stearic Acid | Witco Corp., TX |
| Hampene Na 4 | Ethylene Diamine Tetraacetic Sod. Salt | Hampshire Chem. Corp. |
| Polyglcol E400 | Polyethylene Glycol 400 | D.V.C. Limited Inc., NJ |
| Sodium Stearate C7 | Sodium Stearate | Witco Corp., TX |
| Dow Corning Fluid 344 | cyclomethicone | Dow Corning, MI |
| Dow Corning Fluid 200 | Dimethicone | Dow Corning, MI |
| Carbomer (Ultrez 10) | Carbopol ETD 2001 Resin | B. F. Goodrich, OH |
| Brij 78 | Steareth-20 | ICI, DE |
| Triclosan | Irgason DP 300 | Ciba Geigy, NC |
| Dow Corning Fluid 345 | Cycomethicone | Dow Corning, MI |
| Adol 62 | Stearyl Alcohol | Witco Corp., TX |
| Castor Wax MP 70 | Hydrogenated Castor Oil | Cas Chem., NJ |
| Reach AZP 908 | Aluminum Zirconium Tetrachloro-Gly | Reheis Inc, NJ |
| Silica | Cabosil M-5 | Cabot Corp., IL |
| Germaben II | Diazolidinyl urea | ISP, NJ |
| Escalol 567 | Benzopherone 3 | ISP, NJ |
| Escalol 587 | Octylsalicylate | ISP, NJ |
| Parsol MCX | Octylmethoxycinnomate | Roche Vitamins, NJ |
| Witconol 2314 | Iso Propyl Myristate | Witco Corp., TX |
| Witconol 2316 | Iso Propyl Palmitate | Witco Corp., TX |
| Finsolv SB | Isostearyl Benzoate | Finetex Inc., NJ |
| Finsolv PG22 | Dipropylene Glycol Dibenzoate | Finetex Inc., NJ |
| Finsolv BOD | Octyl Dodecyl Benzoate | Finetex Inc., NJ |
| Ajidew N-50 | Sodium PCA (Pyrrolidone | Ajinomoto |
| | Carboxylic Acid) | Paramus, N.J. |
| Versene Na2 crystals | Disodium EDT | Dow Chem. Co, Midland, MI |
| Standapol ES-40 | Sodium Laureth Sulfate | Cognis, Ambler, PA |
| Pluracare L-64 | Poloxamer 184 | BASF, NJ |
| Makam 2C | Disodium cocamphodiacetate | McIntyre Group |
| | | University park, IL |
| Ritapan DL | Panthenol | R.I.T.A. Chemical |
| | | Woodstock, IL |
| Germal Plus | Diazolidinyl Urea & | ISP, Wayne, NJ |
| | Iodopropynyl Butylcarbamate | |
| Biopol HC | Hyaluronic Acid & | Brooks Industries |
| | Collagen Complex | S. Plainfield, NJ |
| Cocobetaine | Cocoamidopropyl betaine | McIntryre Group |
| | | University Park, IL |
| Versene 100 XL | Tetrasodium EDTA | Dow Chemical Co. |
| | | Midland, MI |
| Mackamide LLM | Lauramide DEA | McIntyre Group |
| | | University Park, IL |
| Glycerox HE | PEG-7-Glycerol Cocoate | Croda, Parsippany, NJ |
| Hamposyl L-30 | sodium lauroyl sarcosinate | Hampshire Chem. Co. |
| | | Lexington, MA |
| Crothix | PEG-150 Pentaerythrityl | Croda, Parsippany, NJ |
| | Tetrastearate | |
| Hydrosoy 2000Sf | hydrolyzed soy protein | Croda, Parsippany, NJ |
| Fragrance 808999 | | Mannheimmer |
| | | Teterboro, NJ |
| Kathon CG | methylchloroisothiazolinone | Rohm & Haas Co. |
| | | Philadelphia, PA |
| Pemulan TR-1 | Acrylates/C10-30 Alkyl | B. F. Goodrich, |
| | Acrylate Cross Polymer | Brecksville, Ohio |
| Natrosol 250HHRCS | Hydroxyethylcellulose | Hercules, |
| | | Wilmingon, DE |
| Standapol A | Ammonium lauryl sulfate | Cognis, Ambler, PA |
| Makam L | Cocoamidopropylbetaine | McIntyre Group, |
| | | University Park, ILL |

We claim:

1. A macadamia-lipid based derivative represented by the following structure 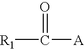
where R₁=macadamia fatty acids group and A is selected from the group consisting of: 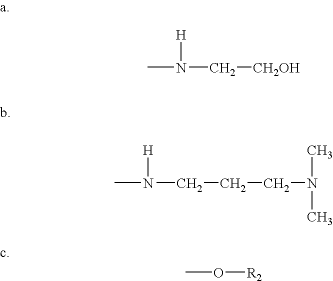
where R₂=IsoStearyl Alcohol Group
where R₂=PPG-3 Myristyl Ether Group
where R₂=C12-15 Pareth-7 Alcohol Group
where R₂=Cetereath-4 Alcohol Group 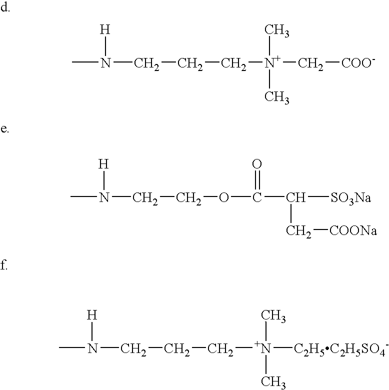
and
g. a blend of the compound of claim 1 and Sodium methyl cocoyl taurate.

2. A macadamia-lipid based derivative which is a mixture, said mixture formed by reacting macadamia fatty triglycerides and monoethanolamine, said macadamia-lipid based derivative comprising macadamia-amide-MEA represented by the following structure: 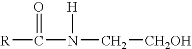
where R=macadamia fatty acids group.

3. An external skin care preparation comprising a macadamia-lipid based derivative as defined in claim 1.

4. A cosmetic hair care formulation comprising a macadamia-lipid based derivative as defined in claim 1.

5. A skin or hair care method comprising applying to the skin or hair a preparation or formulation comprising a macadamia-lipid based derivative as defined in claim 1.

6. A macadamia-lipid based derivative formed by blending monoethanolamide of macadamia fatty triglycerides and Sodium methyl cocoyl taurate,

7. An external skin care preparation comprising a macadamia-lipid based derivative as defined in claim 6.

8. A cosmetic hair care formulation comprising a macadamia-lipid based derivative as defined in claim 6.

9. A skin or hair care method comprising applying to the skin or hair a preparation or formulation comprising a macadamia-lipid based derivative as defined in claim 6.

10. A macadamia-lipid based product which is a mixture, said mixture formed by reacting macadamia fatty triglycerides and dimethylaminopropylamine, said macadamia-lipid based product comprising macadamiaamidopropyldimethylamine represented by the following structure: 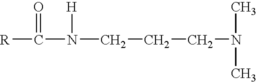
where R=Macadamia Fatty Acids group.

11. An external skin care preparation comprising a macadamia-lipid based product as defined in claim 10.

12. A cosmetic hair care formulation comprising a macadamia-lipid based product as defined in claim 10.

13. A skin or hair care method comprising applying to the skin or hair a preparation or formulation comprising a macadamia-lipid based product as defined in claim 10.

14. A macadamia-lipid based product which is a mixture, said mixture formed by reacting macadamia fatty triglycerides methylated and IsoStearyl Alcohol, said macadamia-lipid based product comprising IsoStearyl Macadamiate represented by the following structure: 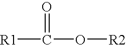
where R₁=Macadamia Fatty Acids group; and
where R₂ is selected from the group consisting of IsoStearyl alcohol, PPG-3 myristyl ether, C12-15 Pareth-7 Alcohol, and Cetereath-4 Alcohol.

15. An external skin care preparation comprising a macadamia-lipid based product as defined in claim 14.

16. A cosmetic hair care formulation comprising a macadamia-lipid based product as defined in claim 14.

17. A skin or hair care method comprising applying to the skin or hair a preparation or formulation comprising a macadamia-lipid based product as defined in claim 14.

18. A macadamia-lipid based product which is a mixture, said mixture formed by reacting macadamia amido propyl dimethyl amine and sodium monochloroacetate, said macadamia-lipid based product comprising macadamia amidopropylbetaine represented by the following structure: 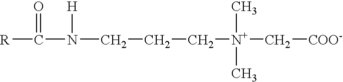
Where R=Macadamia fatty acids group.

19. An external skin care preparation comprising a macadamia-lipid based product as defined in claim 18.

20. A cosmetic hair care formulation comprising a macadamia-lipid based product as defined in claim 18.

21. A skin or hair care method comprising applying to the skin or hair a preparation or formulation comprising a macadamia-lipid based product as defined in claim 18.

22. A macadamia-lipid based product which is a mixture, said mixture formed by reacting Macadamia Amide-MEA-Maleate and sodium sulfite, said macadamia-lipid based product comprising Disodium Macadamia-Amide-MEA-Sulfosuccinate represented by the following structure: 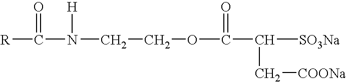
Where R=macadamia fatty acids group.

23. An external skin care preparation comprising a macadamia-lipid based product as defined in claim 22.

24. A cosmetic hair care formulation comprising a macadamia-lipid based product as defined in claim 22.

25. A skin or hair care method comprising applying to the skin or hair a preparation or formulation comprising a macadamia-lipid based product as defined in claim 22.

26. A macadamia-lipid based product which is a mixture, said mixture formed by reacting macadamiaamidopropyl dimethyl amine and diethylsulfate, said macadamia-lipid based product comprising Macadamia-Amidopropyl Dimethyl Ethyl Ethosulfate Quaternary represented by the following structure: 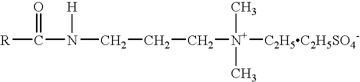
Where R=macadamia fatty acids group.

27. An external skin care preparation comprising a macadamia-lipid based product as defined in claim 26.

28. A hair care formulation comprising a macadamia-lipid based product as defined in claim 26.

29. A skin or hair care method comprising applying to the skin or hair a preparation or formulation comprising a macadamia-lipid based product as defined in claim 26.

30. A composition comprising a derivative of macadamia fatty triglycerides and a vehicle for enabling said composition to be applied to the skin or hair.

31. A method for treating or preventing skin damage, comprising topically applying to the skin an effective amount of a dermatologically acceptable carrier and a macadamia-lipid based derivative.

32. The method of claim 31 wherein said macadamia-lipid based derivative is selected from the group consisting of amides of macadamia fatty triglycerides/monoethanolamine, Diethanolamine, Monoisopropanolamine, Diisopropanolamine, macadamia-amidopropyl dimethyl amines, betaines based on MFTG, esters of macadamia fatty triglycerides and Isostearyl Alcohol; Quaternaries based on macadamia-amidopropyldimethyl amines, Sulfosuccinamates based on macadamia amide MEA-maleate and sodium sulfite, and esters of MFTG and PPG-3 myristyl ether, ceteareth-4, and C12-15 Pareth-7.

33. The method of claim 31 wherein said composition comprises about 1.0% to about 50% by weight of said macadamia-lipid based derivative.

34. The method of claim 31 wherein said composition is formulated as a solution, gel, lotion, cream, ointment or solid stick.

35. A method for cleaning or conditioning hair, comprising topically applying to the hair an effective amount of a composition comprising a denmatologically acceptable carrier and a macadamia-lipid based derivative.

36. The method of claim 35 wherein said macadamia-lipid based derivative is selected from the group consisting of amides of macadamia fatty triglycerides/monoethanolamine, Diethanolamine, Monoisopropanolamine, Diisopropanolamine, macadamia-amidopropyl dimethyl amines, betaines based on MFTG, esters of macadamia fatty triglycerides and Isostearyl Alcohol; Quaternaries based on macadamia-amidopropyldimethyl amines, Sulfosuccinamates based on macadamia amide MEA-maleate and sodium sulfite, and esters of MFTG and PPG-3 myristyl ether, ceteareth-4, and C12-15 Pareth-7.
